(19) United States
(12) Patent Application Publication
(10) Pub. No.: US 2002/0065563 A1
(43) Pub. Date: May 30, 2002

(54) Implantable artificial bladder

(76) Inventors: Roland Gerlach, Guxhagen (DE); Josef Hannappel, Pulheim (DE); Juergen Reuter, Alheim (DE); Dorothea Rohrmann, Langerwehe (DE)

Correspondence Address: Diller Ramik & Wight PC, Merrion Square Suite 101, 7345 McWhorter Place, Annandale, VA, 22003, US

(21) Appl. No.: 09/478,898

(22) Filed: Jan. 7, 2000

(30) Foreign Application Priority Data
Jan. 13, 1999 (DE) 199 00 940.6

(51) Int. Cl. A61F 2/04

(52) U.S. Cl. 623/23.68; 623/23.65

(57) ABSTRACT

The bladder consists of two chambers of self-restoring elastic material. A check valve is provided between both chambers that is biased in the flow direction and allows liquid to pass only when the differential pressure exceeds a certain limit value. The liquid from the kidney first reaches the first chamber, and after a certain amount of liquid has accumulated there, the liquid flows through the valve into the second chamber. The chambers are arranged in an eight-shaped configuration. The bladder has a flat design and allows for an advantageous anatomic adaptation to the implantation area between ribs and ala of the ilium. Further, a suction pressure is obtained that is relatively uniform in regard of the filling level.

### BACKGROUND OF THE INVENTION

The present invention relates to an implantable artificial bladder that is implanted subcutaneously into a patient to receive the urine secreted from the kidneys.

Known artificial bladders consist of an elastic receptacle biased in the direction of widening. One connection of the receptacle is connected to one or both kidneys so that the receptacle draws urine from the kidney. Another connection of the receptacle is connected with the urethra. The receptacle is emptied by manually compressing the receptacle against its elastic force, whereby the liquid within the receptacle is expelled into the urethra. These known bladders are disadvantageous in that the relatively large receptacle, generally a flat round receptacle, is mostly too large for the space available between the ribs and the ala of the ilium and causes inconveniences to the patient. Moreover, the bulge of the receptacle stresses the skin suture at the implantation site.

An artificial bladder must be self-primed, i.e. it has to draw urine from the kidney so as to support the ureter function. The draw-off from the kidney is normally done via the ureter by peristaltic movements of muscles. Usually, a slight excess pressure prevails in the renal pelvis, but preliminary examinations have shown that the renal parenchyma also accepts slight vacuums. If the vacuum is created by an artificial bladder, it is difficult to maintain a uniform vacuum for all filling levels of the artificial bladder. To produce the required suction effect also when the bladder is nearly full, artificial bladders are usually designed such that they produce a much too strong suction effect when empty, whereby damage could be done to the kidney.

### SUMMARY OF THE INVENTION

It is the object of the present invention to provide an implantable artificial bladder that allows for a flat structure and causes less stress to the body.

The bladder of the present invention is provided with two chambers connected by a check valve. Both chambers are biased to their open state so that they cause suction. The chambers are arranged side by side whereby the volume of the chambers is spatially distributed. This structure allows for a better adaptation to the anatomy of the body so that the bladder can be implanted between the ribs and the ala of the ilium without causing inconveniences. Further, as compared to known bladders having only a single chamber, a flatter structure of the same volume is obtained. As a consequence, the skin suture at the implantation site is stressed less. Moreover, the curve of the suction pressure as a function of the filling level of the bladder becomes more constant. Therefore, a lower suction pressure suffices that will not physiologically stress the kidney.

The check valve between the chambers prevents urine from flowing back. It allows the contents of the inlet-side chamber to be expelled into the outlet-side chamber.

Preferably, the suction effects of the two chambers are different, the outlet-side chamber having the largest suction effect. The check valve is biased in the flow direction, opening only when the pressure difference between the two chambers exceeds a limit value. This limit value is higher than the pressure difference that occurs in the chambers as a consequence of different return forces of the chamber walls. When liquid flows into the inlet-side chamber and fills a part of the chamber volume, the pressure difference exceeds the limit value so that liquid flows into the outlet-side chamber until the check valve closes again. Thus, the suction pressure or the vacuum is reduced only relatively slightly as the filling level of the bladder rises.

In a preferred embodiment of the invention, the chambers each have a self-returning first wall and an elastic sheet wall tensioned by the first wall. Thus, each chamber comprises a relatively thick-walled flexibly resilient first wall and an elastically extensible sheet wall that has practically no flexural elasticity.

### BRIEF DESCRIPTION OF THE DRAWINGS

The following is a detailed description of an embodiment of the present invention with reference to the accompanying drawings.

In the Figures

FIG. 1 is a longitudinal section through the artificial bladder,

FIG. 2 is a top plan view on FIG. 1, and

FIG. 3 shows a schematic course of the vacuum/volume diagram of the bladder.

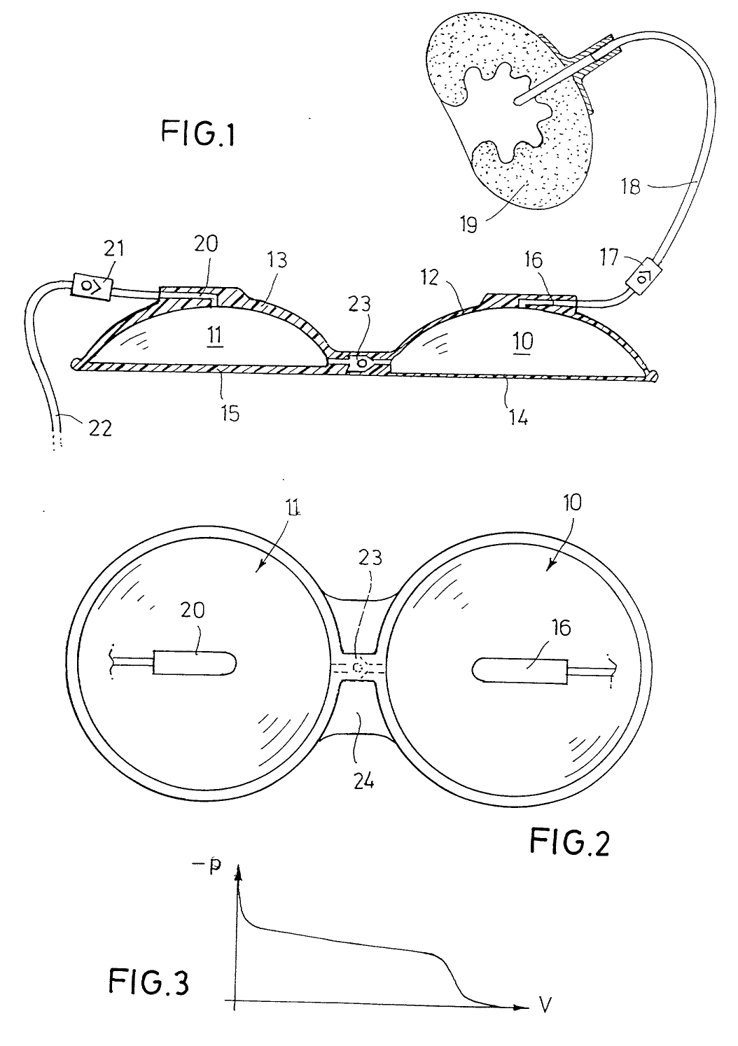

The illustrated artificial bladder is made of an elastic plastic material well tolerated by the body, in particular silicone material. It is generally eight-shaped in plan view, the shape being formed by two side by side circular chambers 10, 11. The chamber 10 has a convexly arched upper wall 12, the wall thickness thereof being greatest in the center (in the apex region) and decreasing towards the lateral edges. The chamber 11 has an upper wall 13 of the same design as the upper wall 12, the wall thickness being greater, however, so that a stronger suction effect is created in the chamber 11 than in the chamber 10.

Each chamber 10, 11 has a bottom formed by a sheet wall 14, 15, respectively. The sheet walls 14 and 15 merge and consist of an integral sheet. The circumferential edges of the upper walls 12, 13 are glued or welded to the sheet walls 14, 15 so that the chambers 10, 11 are hermetically sealed to the outside.

At the apex of the upper wall 12, the chamber 10 has a connection 16 to which an external hose may be connected, the connection 16 is provided with a check valve 17 which is connected to a kidney 19 via a hose 18 and opens only into the chamber 10, blocking the hose in the opposite direction.

The upper wall 13 of the chamber 11 is provided with a connection 20 connected with a check valve 21 opening only in the outlet direction, i.e., out from the chamber 11. A hose 22 leads from the check valve 21 to the urethra (not illustrated).

In the representation of FIG. 2 the check valves 17 and 21 are integrated in the respective connections 16 and 20. Both chambers 10, 11 are connected by a joining web 2. They form a bladder shaped like a lying “8”.

A check valve 23 is provided between both chambers 10, 11 that allows flow only from the chamber 10 to the chamber 11, yet blocks the flow in the opposite direction. The check valve is a pre-loaded check valve opening only when the differential pressure exceeds a certain limit value.

In the empty state of the chambers, the respective sheet wall 14 or 15 abuts the inner side of the upper wall 12 or 13, the chamber volume being reduced to zero. Together with the elasticity of the sheet wall 14, 15, the elasticity of the upper wall determines the restoring force or the magnitude of the suction force exerted by the chamber. The suction characteristics of the bladder can be altered by the selection of a suitable material of the sheet wall 14, 15, while using the same upper wall 12, 13.

Since the wall thickness of the upper wall 13 of the second chamber 11 is greater than that of the upper wall 12 of the first chamber, the second chamber produces a stronger suction effect than the first chamber. Yet, the differential pressure is lower than the limit value at which the check valve 23 opens.

The first chamber 10 draws urine from the kidney 19 with the lower suction force. When the chamber 10 is partly filled with liquid, the check valve 23 opens so that a part of this liquid gets into the second chamber 10. In this manner, both chambers are filled subsequently, the liquid always reaching the chamber 10 first and a part of the liquid being transferred into the chamber 11 afterwards. When both chambers 10, 11 are full, the liquid is pressed out by manual pressure on the chamber 11 or by pumping action. Upon release of pressure, liquid is again taken into the chamber 11 from chamber 10 and pressed out from the chamber 11 by exerting pressure thereon.

FIG. 3 schematically illustrates the course of the vacuum -p as a function of the liquid volume v contained in the bladder. It is obvious that the curve is rather shallow, i.e. the suction pressure changes relatively little as a function of the liquid volume.

What is claimed is:

1. An implantable artificial bladder with at least two compressible chambers biased in the open state, each having a connection, said chamber being connected through a check valve.

2. The bladder of claim 1, wherein the chambers are arranged side by side in an eight-shaped configuration.

3. The bladder of claim 1, wherein the chambers each have a self-restoring first wall and an elastic sheet wall tensioned by the first wall.

4. The bladder of claim 1, wherein the check valve between the chambers is biased in the flow direction and opens when the differential pressure exceeds a limit value.

5. The bladder of claim 1, wherein the downstream chamber has a stronger suction effect than the upstream chamber.
